SEQUENCE LISTING (2 sequences)

SEQ ID NO: 1 - DNA, 4329 bp, Zea mays
  CDS (2)..(4009)
cccagatggcacgacagtgtacaagaacagggctctcaggacaccttttgtaaagtcaggtgataactccactctaaggattgagatagatgatcctggggtgcacgccattgagttcctcatctttgacgagacacagaacaaatggtttaaaaacaatggccagaattttcaggttcagttccagtcgagccgccatcagggtactggtgcatctggtgcctcctcttctgctacttctaccttggtgccagaggatcttgtgcagatccaagcttaccttcggtgggaaagaaggggaaagcagtcatacacaccagagcaagaaaaggaggagtatgaagctgcacgagctgagttaatagaggaagtaaacagaggtgtttctttagagaagcttcgagctaaattgacaaaagcacctgaagcacccgagtcggatgaaagtaaatcttctgcatctcgagtgcccatcggtaaacttccagaggatcttgtacaggtgcaggcttatataaggtgggagcaagcgggcaaaccaaactatcctcctgagaagcaactggtagaatttgaggaagcaaggaaggaactgcaggctgaggtggacaagggaatctctattgatcagttgaggcagaaaattttgaaaggaaacattgagagtaaagtttccaagcagctgaagaacaagaagtacttctctgtagaaaggattcagcgcaaaaagagagatatcacacaacttctcagtaaacataagcatacagttatggaagataaagtagaggttgtaccaaaacaaccaactgttcttgatctcttcaccaagtctttacatgagaaggatggctgtgaagttctaagcagaaagctcttcaagttcggcgataaagagatactggcaatttctaccaaggttcaaaataaaacagaagttcacttggcaacaaaccatacggacccacttattcttcactggtctttggcaaaaaatgctggagaatggaaggcaccttctccaaatatattgccatctggttccacattgctggacaaggcgtgtgaaactgaatttactaaatctgaattggatggtttgcattaccaggttgttgagatagagcttgatgacggaggatacaaaggaatgccatttgttcttcggtctggtgaaacatggataaaaaataatggttctgattttttcctagatttcagcacccatgatgtcagaaatattaaggcaattttaaaggacaatggcgatgctggtaaaggtacttctaaggcgttgctggagagaatagcagatctggaggaagatgcccagcgatctcttatgcacagattcaatattgcagcagatctagctgaccaagccagagatgctggacttttgggtattgttgggctttttgtttggattagattcatggctaccaggcaactaacatggaataagaactataatgtgaagccacgtgagataagcaaagcacaggataggtttacagatgatcttgagaatatgtacaaaacttatccacagtacagagagatattaagaatgataatggctgctgttggtcgcggaggtgaaggtgatgttggtcaacgcattcgtgatgagatattagtaatacagagaaataatgactgcaaaggtggaatgatggaagaatggcaccagaaattgcacaacaatacaagcccagatgatgtagtgatatgccaggccttaattgattatatcaagagtgactttgatataagcgtttactgggacaccttgaacaaaaatggcataaccaaagagcgtctcttgagctatgatcgtgctattcattcagaaccaaatttcagaagtgaacagaaggcgggtttactccgtgacctgggaaattacatgagaagcctaaaggctgtgcattctggtgctgatcttgaatctgctatagcaagttgtatgggatacaaatcagagggtgaaggtttcatggttggtgttcagatcaatccagtgaagggtttaccatctggatttccggagttgcttgaatttgtgcttgaacatgttgaggataaatcagcggaaccacttcctgaggggctattggaagctcgagttgaactgcgccctttgcttcttgattcgcgtgaacgcatgaaagatcttatatttttggacattgctcttgattctaccttcaggacagcaattgaaaggtcatatgaggagctgaatgatgcagccccagagaaaataatgtacttcatcagtcttgtccttgaaaatcttgcgctttcaattgacgacaatgaagacatcctgtattgtttaaagggatggaaccaagccttggaaatggctaagcaaaaagacgaccaatgggcgctctatgctaaagcatttcttgacagaaacagacttgcccttgcgagcaagggagaacaataccataatatgatgcagccctctgctgagtatcttggctcgttactcagcatagaccaatgggcagtcaatatcttcacagaagaaattatacgcggtggatcagctgctactctgtctgctcttctgaaccgatttgatcctgttttaaggaatgttgctcacctcggaagttggcaggttataagcccggttgaagtatcaggttatgtggttgtggttgatgagttacttgctgtccagaacaaatcttatgataaaccaaccatccttgtggcaaagagtgtcaagggagaggaagaaataccagatggagtagttggtgtaattacacctgatatgccagatgttctgtctcatgtgtcagtccgagcaaggaatagcaaggtactgtttgcgacctgttttgaccacaccactctatctgaacttgaaggatatgatcagaaactgttttccttcaagcctacttctgcagatataacctatagggagatcacagagagtgaacttcagcaatcaagttctccaaatgcagaagttggccatgcagtaccatctatttcattggccaagaagaaatttcttggaaaatatgcaatatcagccgaagaattctctgaggaaatggttggggccaagtctcggaatatagcatacctcaaaggaaaagtaccttcatgggtcggtgtcccaacgtcagttgcgataccatttggcacttttgagaaggttttgtcagatgggcttaataaggaagtagcacagagcatagagaagcttaagatcagacttgcccaagaagattttagtgctctaggtgaaataagaaaagtcgtccttaatcttactgctcctatgcaattggttaatgagctgaaggagaggatgctaggctctggaatgccctggcctggtgatgaaggagacaagcgttgggagcaagcatggatggctattaaaaaggtttgggcatcaaaatggaacgaaagagcatattttagcacacgcaaggtgaaacttgatcatgagtacctttcgatggctgttctcgtgcaagaagttgtgaatgcagattatgcttttgtcattcataccacaaacccatcgtctggagattcttctgagatatatgctgaagtggtgaaagggcttggcgagaccctcgtgggagcctatcctggtcgtgctatgagctttgtttgcaaaaaagatgaccttgactctcccaagttacttggttacccaagcaagccaattggtctcttcataaggcaatcaatcatcttccgttccgactccaacggtgaggacctggaaggttatgctggagcaggattatatgatagtgtaccgatggatgaggaggatgaggttgtacttgattatacaactgaccctcttatagtagaccgtggattccgaagctcaatcctctcaagcatagcacgggctggccatgccatcgaggagctatatggttctcctcaggacgtcgagggagtagtgaaggatggaaaaatctatgtagtccagacaagaccacagatgtagtatgtatgcatctattagacagctcaataagcactgttgtacgcttgtatggttgggacatatgggcgttatggcatgtatagttgtatgcctagatgtacaacacgtgtactcgtatatatatatataaatgctgaaacaagcattggtcctgtactgtagtttctacatttcattgtcaccaataattaagtgtactcctatggctgggagtctatgaaaatggacgtgttgacttattgggtaataaataatttatatataaaaaaaaaaaaaaaaaaaaaaaaaaaaaaaaaaaaaaaaaaaaaaaaaaaaaa

SEQ ID NO: 2 - protein, 1336 aa, Zea mays
PDGTTVYKNRALRTPFVKSGDNSTLRIEIDDPGVHAIEFLIFDETQNKWFKNNGQNFQVQFQSSRHQGTGASGASSSATSTLVPEDLVQIQAYLRWERRGKQSYTPEQEKEEYEAARAELIEEVNRGVSLEKLRAKLTKAPEAPESDESKSSASRVPIGKLPEDLVQVQAYIRWEQAGKPNYPPEKQLVEFEEARKELQAEVDKGISIDQLRQKILKGNIESKVSKQLKNKKYFSVERIQRKKRDITQLLSKHKHTVMEDKVEVVPKQPTVLDLFTKSLHEKDGCEVLSRKLFKFGDKEILAISTKVQNKTEVHLATNHTDPLILHWSLAKNAGEWKAPSPNILPSGSTLLDKACETEFTKSELDGLHYQVVEIELDDGGYKGMPFVLRSGETWIKNNGSDFFLDFSTHDVRNIKAILKDNGDAGKGTSKALLERIADLEEDAQRSLMHRFNIAADLADQARDAGLLGIVGLFVWIRFMATRQLTWNKNYNVKPREISKAQDRFTDDLENMYKTYPQYREILRMIMAAVGRGGEGDVGQRIRDEILVIQRNNDCKGGMMEEWHQKLHNNTSPDDVVICQALIDYIKSDFDISVYWDTLNKNGITKERLLSYDRAIHSEPNFRSEQKAGLLRDLGNYMRSLKAVHSGADLESAIASCMGYKSEGEGFMVGVQINPVKGLPSGFPELLEFVLEHVEDKSAEPLPEGLLEARVELRPLLLDSRERMKDLIFLDIALDSTFRTAIERSYEELNDAAPEKIMYFISLVLENLALSIDDNEDILYCLKGWNQALEMAKQKDDQWALYAKAFLDRNRLALASKGEQYHNMMQPSAEYLGSLLSIDQWAVNIFTEEIIRGGSAATLSALLNRFDPVLRNVAHLGSWQVISPVEVSGYVVVVDELLAVQNKSYDKPTILVAKSVKGEEEIPDGVVGVITPDMPDVLSHVSVRARNSKVLFATCFDHTTLSELEGYDQKLFSFKPTSADITYREITESELQQSSSPNAEVGHAVPSISLAKKKFLGKYAISAEEFSEEMVGAKSRNIAYLKGKVPSWVGVPTSVAIPFGTFEKVLSDGLNKEVAQSIEKLKIRLAQEDFSALGEIRKVVLNLTAPMQLVNELKERMLGSGMPWPGDEGDKRWEQAWMAIKKVWASKWNERAYFSTRKVKLDHEYLSMAVLVQEVVNADYAFVIHTTNPSSGDSSEIYAEVVKGLGETLVGAYPGRAMSFVCKKDDLDSPKLLGYPSKPIGLFIRQSIIFRSDSNGEDLEGYAGAGLYDSVPMDEEDEVVLDYTTDPLIVDRGFRSSILSSIARAGHAIEELYGSPQDVEGVVKDGKIYVVQTRPQM

(19) United States
(12) Patent Application Publication
(10) Pub. No.: US 2006/0150278 A1
(43) Pub. Date: Jul. 6, 2006

(54) Method for generating maize plants with an increased leaf starch content, and their use for making maize silage

(75) Inventors: Claus Frohberg, Berlin (DE); Michael Bauerlein, Berlin (DE)

Correspondence Address: BIRCH STEWART KOLASCH & BIRCH, PO BOX 747, FALLS CHURCH, VA, 22040-0747, US

(73) Assignee: PlantTec Biotechnologie GmbH Forschung & Entwicklung, Potsdam (DE)

(21) Appl. No.: 10/371,078

(22) Filed: Feb. 21, 2003

(30) Foreign Application Priority Data
Feb. 26, 2002 (DE) 10208132.8

(51) Int. Cl. A01H 1/00; A01H 5/00; C12N 15/82

(52) U.S. Cl. 800/284; 800/288; 800/320.1; 435/468

(57) ABSTRACT

There is described a method for generating transgenic maize plants with an increased leaf starch content. Such plants are suitable for making an improved silage. There are furthermore described the use of the transgenic maize plants for making silage, and suitable ensiling methods using such plants.

The present invention relates to a method for generating transgenic maize plants which are genetically modified, the genetic modification in the cells or plants bringing about the reduced activity of an R1 protein in comparison to corresponding wild-type plant cells or plants which have not been genetically modified. Maize plants generated by this method synthesize a modified starch, the starch content in the leaves of the plants being up to 1 000% higher than that of wild-type plants which have not been genetically modified. This starch is furthermore preferably characterized in that it has a reduced phosphate content. Furthermore, the present invention relates to a method for making an improved maize silage in which the maize plants generated by the method according to the invention are used, and to the silage obtainable by the method.

The quality and composition of silage influence the consumption capacity, the digestibility and the conversion rate in animal feeding, for example in beef production and sheep fattening. The quality of each silage depends predominantly on the ensiled plants. Depending on what the agricultural enterprise focuses on, a variety of plants are suitable for ensiling. Conventionally used silage plants are mainly maize (in the form of corn-cob mix (CCM)=dehusked maize ears which are chopped and ensiled directly) or corn silage (CS=all of the plant is chopped) and grasses (such as, for example, Lolium) and sugar beet (sliced or in the form of beet tops). Only animals are capable of converting silage properly. The farmer expects that an improved feed quality silage will lead to better conversion of the material fed and that the energy requirements in livestock feeding will be met more specifically. However, the differences between ruminants and non-ruminants must be taken into consideration. In cattle, the supplementation with concentrates only meets part of the energy requirements since the specific digestion processes in ruminants require a minimum of structured roughage. Silage plants should therefore have a high utilizable energy content and ensure very good consumption. High energy requirement is in proportion to digestibility. Consumption depends on the rate of passage through the gut. Not only the quality, but also the age of the ensiled plants has a pronounced impact on the utilization of the silage in the animal's digestion process.

Fodder plants, such as fodder legumes and fodder grasses, are very high in protein, but only contain a small amount of non-soluble carbohydrates. As a consequence, most of the ammonium produced cannot be metabolized, but, in order to avoid poisoning, is transported to the liver, converted into uric acid and then excreted. This leads to nitrogen loss and simultaneously large amounts of excreted ammonia.

It is sought to avoid this by using silage plants with an improved carbon/nitrogen ratio (C/N) and, as a consequence, better digestibility and energy intake and, eventually, higher production of meat/milk in combination with less excreted ammonia.

Besides individual amino acids, trace elements and the like, readily fermentable carbohydrates are important for the microbial in-rumen protein synthesis and thus for the protein supply of the animals. A higher percentage of readily digestible carbohydrates results in more rapid degradation in the rumen. This brings about lower pH values, smaller amounts of acetic acid and less digestion of dietary fibre, plus a higher percentage of propionic acid and increasing microbial protein synthesis. Since nutrient conversion in the small intestine exceeds that in the rumen, but the small intestine only has a limited capacity for starch hydrolysis, the absorption of glucose and the microbial fermentation of glucose (Lebzien et al., Kraftfutter/Feed Magazine (2001), 356-366), improved in-rumen utilization of the silage is of particular importance.

The present invention is based on the object of providing a method by means of which plants which have a much higher utilizable energy content and which are thus suitable for making a silage which is capable of better utilization by animals, in particular ruminants, can be generated.

This object is achieved by providing the use forms specified in the patent claims.

The present invention thus relates to a method for generating transgenic maize plants with an increased leaf starch content in comparison to corresponding wild-type plants, where
(a) a cell of a maize is genetically modified by introducing a foreign nucleic acid molecule whose presence or expression leads to a reduced activity of an R1 protein which occurs endogenously in the plant;
(b) a plant is regenerated from the cell generated in step (a); and
(c) if appropriate, further plants are generated starting from the plant generated in step (b).

In the present context, the term “transgenic” means that the plants generated by the method according to the invention differ, owing to a genetic modification, in particular the introduction of a foreign nucleic acid molecule, with regard to their genetic information from corresponding plant cells which have not been genetically modified and can be distinguished from them. In particular, the term means that the cells of these plants contain a foreign nucleic acid molecule which does not occur naturally in corresponding wild-type plants which have not been genetically modified or which occurs at a locus in the genome of the cells at which it does not occur naturally. In this context, the term “wild-type plants” means a maize plant (Zea mays) which, however, contains no corresponding genetic modification, in particular no genetic modification in connection with the R1 gene. In this context, “foreign” nucleic acid molecule means that the nucleic acid molecule is heterologous with regard to maize, or that, if the nucleic acid molecule is homologous to maize, it occurs in the genetically modified cell in a genetic context in which it does not occur naturally in the plant cells. This means that it occurs at a different locus in the genome of the plant cell and/or is linked to sequences to which it is not linked naturally in the plant cells. Whether a plant or plant cell is transgenic can be verified by methods with which the skilled worker is familiar, for example Southern blot analysis.

The term “maize plant” is understood as meaning a maize plant which belongs to the species Zea mays.

The term “increased starch content” refers to the fact that the amount of starch formed in the leaves markedly exceeds the amount formed in the leaves of corresponding wild-type plants. The starch content of the maize plants generated by the method according to the invention is increased by at least 10-50%, preferably by at least 50-100%, in particular by 100-500% and very particularly preferably by at least 500-1 000% in comparison with the starch content of corresponding wild-type plants.

The starch content in the leaf is preferably measured in nmol/g dry matter. Methods for determining the starch content are known to the skilled worker and described for example in the examples which follow.

For the purposes of the present invention, the term “genetically modified” refers to the fact that the genetic information of the plant cell is modified in comparison with corresponding cells of a wild-type plant by the introduction of a foreign nucleic acid molecule and that the presence and/or the expression of the foreign nucleic acid molecule results in an altered phenotype of the maize plant regenerated from this plant cell. In this context, altered phenotype preferably means a measurable difference of one or more functions of the cells and/or the plant. By way of example, in plant cells which have been genetically modified in accordance with the method of the invention, the activity of an R1 protein which occurs endogenously in the plant cell is reduced in comparison with corresponding plant cells of wild-type plants which have not been genetically modified.

For the purposes of the present invention, the term “reduced activity” refers to a reduced expression of endogenous genes which encode R1 proteins, and/or to a reduced amount of R1 protein in the cells and/or a reduced biological activity of the R1 proteins in the cells.

In this context, “reduced expression” refers to the fact that the maize plants generated by the method according to the invention, or the cells of these plants, contain fewer transcripts which encode an R1 protein than corresponding wild-type plant cells. “Reduced expression” can be determined for example by measuring the amount of transcripts encoding R1 proteins, for example by Northern blot analysis or RT-PCR. Reduction in this context preferably means a reduced amount of transcripts by at least 50%, in particular by at least 70%, preferably by at least 85% and especially preferably by at least 95% in comparison with corresponding cells which have not been genetically modified. In a very especially preferred embodiment, the reduction amounts to 100%, i.e. the expression of R1 genes in the plants (plant cells) is completely repressed, and no R1 protein whatsoever is synthesized in the cells.

A “reduced amount” of R1 protein means that the content of R1 protein in the plants or in the cells of the maize plants which are generated by the method according to the invention is less than in corresponding wild-type plants or wild-type plant cells. Methods for determining the R1 protein content are known to the skilled worker.

Thus, the reduced amount of R1 proteins can be determined for example by Western blot analysis. In this context, reduction means a reduced amount of R1 proteins by at least 50%, in particular by at least 70%, preferably by at least 85%, especially preferably by at least 95% and very especially preferably by 100% in comparison with corresponding wild-type plants or cells which have not been genetically modified.

For the purposes of the present invention, the term “R1 gene” is understood as meaning a nucleic acid sequence (for example RNA or DNA, such as cDNA or genomic DNA) which encodes an “R1 protein”.

For the purposes of the present invention, the term “R1 protein” is understood as meaning proteins which have been described for example in Lorberth et al. (Nature Biotech. 16 (1998), 473-477) and in the international Patent Applications WO 98/27212, WO 00/77229 and WO 00/28052 and which have specific traits. Important traits of R1 proteins are
(i) their localization in the plastids (such as chloroplasts, amyloplasts) of plant cells;
(ii) their property of occurring in the plastids, partly in free form and partly bound to starch granules;
(iii) their ability of influencing the degree of starch phosphorylation in plants, in as much as an increased activity of the R1 protein in plants leads to an increased phosphate content of the starch synthesized in the plants, and a reduced activity of the R1 protein in plants leads to a reduced phosphate content of the starch synthesized in the plants. In this context, the phosphate content relates to the C-6-phosphate content. It is preferably indicated as nmol/mg dry starch and can preferably be determined as described in the examples; and
(iv) their ability of, when expressed in E. coli cells, leading to phosphorylation of the bacterial glycogen. This ability can be assayed for example as described in WO 98/27212.

For the purposes of the present invention, an R1 protein is preferably understood as meaning a protein which has the abovementioned characteristics and which is encoded by a nucleic acid molecule encompassing a nucleotide sequence which hybridizes with the coding region of SEQ ID NO: 1 or its complementary strand. In this context, the term “hybridization” refers to hybridization under conventional hybridization conditions, preferably under stringent conditions as are described, for example, in Sambrook et al. (1989, Molecular Cloning, A Laboratory Manual, 2nd ed. Cold Spring Harbor Laboratory Press, Cold Spring Harbor, N.Y.). Such hybridizing nucleic acid molecules can be isolated for example from genomic libraries or from cDNA libraries of plants, in particular from maize plants.

Such nucleic acid molecules can be identified and isolated using the nucleic acid molecules shown in SEQ ID NO: 1 or parts of these molecules or the reverse complements of these molecules, for example by means of hybridization by standard methods (see, for example, Sambrook and Russel, Molecular Cloning, A Laboratory Manual, 3rd ed. Cold Spring Harbor Laboratory Press (2001), Cold Spring Harbor, N.Y.).

Examples of nucleic acid molecules which can be used as hybridization probe are those with exactly or essentially the sequence shown under SEQ ID NO: 1 or parts of this sequence. The DNA fragments used as hybridization probe may also take the form of synthetic DNA fragments which have been generated with the aid of the customary DNA synthesis techniques and whose sequence agrees essentially with the nucleic acid sequence shown in SEQ ID NO: 1.

“Hybridization” preferably means that at least 60%, preferably at least 80%, especially preferably at least 90% and very especially preferably at least 95% homology, i.e. sequence identity, exists between the molecules in question.

The degree of homology is determined by comparing the nucleotide sequence in question with the coding region shown in SEQ ID NO: 1. If the sequences to be compared differ in length, the degree of homology preferably relates to the percentage of nucleotides in the shorter sequence which are identical to nucleotides in the longer sequence. The homology can be determined by customary methods, in particular using computer programs such as, for example, the DNASTAR program in combination with Clustal W analysis. This program is obtainable from DNASTAR, Inc. 1228 South Park Street, Madison, Wis. 53715 or from DNASTAR, Ltd., Abacus House, West Ealing, London W13 0AS UK (support@dnastar.com), and is accessible via the EMBL server.

If the Clustal analytical method is used for determining homology, in particular for determining whether a sequence is identical, for example 80% identical, to a reference sequence, the settings are preferably as follows: Matrix: blosum 30; Open gap penalty: 10.0; Extend gap penalty: 5.0; Delay divergent: 40; Gap separation distance 8.

The generation of the plant cells with reduced R1 activity in accordance with the method according to the invention can be achieved by various methods with which the skilled worker is familiar, for example by those which lead to inhibition of the expression of endogenous genes which encode an R1 protein. These include, for example, the expression of a corresponding antisense RNA, the provision of molecules or vectors which confer a cosuppression effect, the expression of a suitably constructed ribozyme which specifically cleaves transcripts encoding an R1 protein, or what is known as “in-vivo mutagenesis”.

Furthermore, reduced R1 activity in the plant cells can also be brought about by the simultaneous expression of sense and antisense RNA molecules of the target gene in question which is to be repressed, preferably of the R1 gene. Moreover, it is known that the formation, in planta, of RNA duplexes of promoter sequences in trans can lead to methylation and transcriptional inactivation of homologous copies of this promoter (Mette et al., EMBO J. 19 (2000), 5194-5201).

Furthermore, the use of introns, i.e. of non-coding regions of genes which encode R1 proteins, is also feasible for achieving an antisense or a cosuppression effect. The use of intron sequences for inhibiting the gene expression of genes which encode proteins of starch biosynthesis has been described for example in international Patent Applications WO 97/04112, WO 97/04113, WO 98/37213 and WO 98/37214.

In a preferred embodiment of the method according to the invention, the said foreign nucleic acid molecule is selected from the group consisting of
(a) DNA molecules which encode at least one antisense RNA which brings about reduced expression of endogenous genes which encode R1 proteins;
(b) DNA molecules which, via a cosuppression effect, lead to reduced expression of endogenous genes which encode R1 proteins;
(c) DNA molecules which encode at least one ribozyme which specifically cleaves transcripts of endogenous genes which encode R1 proteins;
(d) nucleic acid molecules which, in the event of in vivo mutagenesis, lead to a mutation or an insertion of a heterologous sequence in endogenous genes which encode R1 proteins, the mutation or insertion leading to reduced expression of genes encoding R1 proteins, or to a reduced synthesis of R1 proteins;
(e) DNA molecules which simultaneously encode at least one antisense RNA and at least one sense RNA, where the said antisense RNA and the said sense RNA form an RNA duplex which brings about a reduced expression of endogenous genes which encode R1 proteins; and
(f) parts or fragments of the nucleic acid molecules or DNA molecules mentioned under a) to d), selected from the group consisting of fragments with at least 25, 50, 100 bp in length, preferably with at least 250 bp in length, especially preferably with at least 500 bp in length.

The skilled worker knows how to achieve an antisense effect or a cosuppression effect. The method of antisense inhibition is described, for example, in Krol et al. (Nature 333 (1988), 866-869), Krol et al. (Gene 72 (1988), 45-50), Mol et al. (FEBS Letters 268 (2), (1990), 427-430), Smith et al. (Plant Mol. Biol. 14 (1990), 369-379) and Sheehy et al. (Proc. Natl. Acad. Sci. USA 85 (1988), 8805-8809). The method of cosuppression inhibition has been described, for example, in Jorgensen (Trends Biotechnol. 8 (1990), 340-344), de Carvalho Niebel et al. (Curr. Top. Microbiol. Immunol. 197 (1995), 91-103), Flavell et al. (Curr. Top. Microbiol. Immunol. 197 (1995), 43-56), Palauqui and Vaucheret (Plant. Mol. Biol. 29 (1995), 149-159), Vaucheret et al. (Mol. Gen. Genet. 248 (1995), 311-317) and in de Borne et al. (Mol. Gen. Genet. 243 (1994), 613-621).

The expression of ribozymes for reducing the activity of certain enzymes in cells is likewise known to the skilled worker and described, for example, in EP-B1 0321 201. The expression of ribozymes in plant cells has been described, for example, in Feyter et al. (Mol. Gen. Genet. 250 (1996), 329-338).

Furthermore, the reduced R1 activity in the plant cells can also be achieved, as mentioned above, by what is known as “in-vivo mutagenesis”, where a chimeric RNA-DNA oligonucleotide (“chimeroplast”) is introduced into cells by cell transformation (Beetham, Kipp et al., Poster Session at the “5th International Congress of Plant Molecular Biology”, 21st-27th Sep. 1997, Singapore; Dixon and Arntzen, Meeting report on “Metabolic Engineering in Transgenic Plants”, Keystone Symposia, Copper Mountain, Colo., USA, TIBTECH 15 (1997), 441-447; international Patent Application WO 95/15972; Kren et al., Hepatology 25 (1997), 1462-1468; Cole-Strauss et al., Science 273 (1996), 1386-1389).

Part of the DNA component of the RNA-DNA oligonucleotide is homologous to a nucleic acid sequence of an endogenous R1 gene, but contains a mutation in comparison with the nucleic acid of an endogenous R1 gene or contains a heterologous region which is flanked by the homologous regions. Owing to base pairing of the homologous regions of the RNA-DNA oligonucleotide and of the endogenous nucleic acid molecule, followed by homologous recombination, the heterologous region or the mutation contained in the DNA component of the RNA-DNA oligonucleotide can be transferred into the genome of a plant cell. The mutation is chosen in such a way that, upon expression of the corresponding sequence, it leads to a reduced activity of an R1 protein, i.e. preferably to a reduced expression of the gene, to a reduced synthesis of an R1 protein or to a reduced biological activity of an R1 protein, for example owing to the expression of inactive proteins, in particular of dominant-negative mutants.

Furthermore, the reduced R1 activity in the plant cells may also be brought about by the simultaneous expression of sense and antisense RNA molecules of the target gene to be repressed, i.e. of the R1 gene. This can be achieved for example by using chimeric constructs which contain inverted repeats of the target gene to be repressed, or of parts of the target gene. In this context, the chimeric constructs encode sense and antisense RNA molecules of the target gene. Sense and antisense RNA are synthesized simultaneously in planta as an RNA molecule, it being possible for sense and antisense RNA to be separated from each other by a spacer, forming an RNA duplex. It has been demonstrated that the introduction of inverted-repeat DNA constructs into the genome of plants is a highly effective method of repressing the genes which correspond with the inverted-repeat DNA constructs (Waterhouse et al., Proc. Natl. Acad. Sci. USA 95 (1998), 13959-13964; Wang and Waterhouse, Plant Mol. Biol. 43 (2000), 67-82; Singh et al., Biochemical Society Transactions 28, part 6 (2000), 925-927; Liu et al., Biochemical Society Transactions 28, part 6 (2000), 927-929); Smith et al., Nature 407 (2000), 319-320; international Patent Application WO 99/53050). Sense and antisense sequences of the target gene(s) may also be expressed separately from one another by means of identical or different promoters (Nap et al., 6th International Congress of Plant Molecular Biology, Quebec, 18th-24th Jun. 2000; Poster S7-27, paper Session S7).

Thus, reduced R1 activity in the plant cells can also be achieved by generating RNA duplexes of R1 genes. For this purpose, it is preferred to introduce, into the genome of plants, inverted repeats of DNA molecules of R1 genes or R1 cDNAs, the DNA molecules to be transcribed (R1 gene or R1 cDNA or fragments thereof) being under the control of a promoter which governs the expression of the said DNA molecules.

Moreover, it is known that the formation of RNA duplexes of promoter DNA molecules in plants in trans can lead to methylation and a transcriptional inactivation of homologous copies of these promoters, which are hereinbelow referred to as target promoters (Mette et al., EMBO J. 19 (2000), 5194-5201). Thus, it is possible, via the inactivation of the target promoter, to reduce the gene expression of a particular target gene (for example of the R1 gene) which is naturally under the control of this target promoter. This means that the DNA molecules which comprise the target promoters of the genes to be repressed (target genes) are now not used as control elements for expressing genes or cDNAs, but as transcribable DNA molecules themselves, which is in contrast to the original function of promoters in plants.

Constructs which are used for generating the target promoter RNA duplexes in planta, where they may exist in the form of RNA hairpin molecules, are preferably those comprising inverted repeats of the target promoter DNA molecules, the target promoter DNA molecules being under the control of a promoter which governs the gene expression of the said target promoter DNA molecules. These constructs are subsequently introduced into the genome of plants. In planta, the expression of the inverted repeats of the said target promoter DNA molecules leads to the formation of target promoter RNA duplexes (Mefte et al., EMBO J. 19 (2000), 5194-5201), by which the target promoter can be inactivated.

The promoter regions of the R1 genes from the plant species in question can be isolated and characterized by screening suitable genomic DNA libraries. Known cDNA or genomic fragments of the R1 genes can be used as homologous probes in this context. The generation and screening of genomic DNA libraries is known to the skilled worker and described in Sambrook and Russell (Molecular Cloning, 3rd edition (2001), Cold Spring Harbor Laboratory Press, NY).

Furthermore, the skilled worker knows that the reduced activity of one or more R1 proteins can be achieved by the expression of non-functional derivatives, in particular trans-dominant mutants of such proteins, and/or by the expression of antagonists/inhibitors of those proteins. Antagonists/inhibitors of those proteins encompass for example antibodies, antibody fragments or molecules with similar binding properties. For example, a cytoplasmic scFv antibody was employed for modulating the activity of the phytochrome A protein in genetically modified tobacco plants (Owen, Bio/Technology 10 (1992), 790-794; Review: Franken et al., Current Opinion in Biotechnology 8 (1997), 411-416; Whitelam, Trends Plant Sci. 1 (1996), 268-272).

In an especially preferred embodiment of the method according to the invention, the gene which occurs endogenously in the fodder plant and which encodes an R1 protein is selected from the group consisting of:
(a) nucleic acid molecules which encompass the coding region of the nucleotide sequence shown in SEQ ID NO: 1;
(b) nucleic acid molecules which encode a protein encompassing the amino acid sequence shown in SEQ ID NO: 2;
(c) nucleic acid molecules whose sequence has at least 80% homology with the nucleic acid molecules mentioned under (a) or (b); and
(d) nucleic acid molecules whose sequence is degenerated in comparison with the sequences of the nucleic acid molecules mentioned under (a), (b) or (c) owing to the genetic code.

A multiplicity of techniques is available for introducing the foreign nucleic acid molecule into a plant host cell in accordance with step (a) of the method according to the invention. These techniques encompass the transformation of plant cells with T-DNA using Agrobacterium tumefaciens or Agrobacterium rhizogenes as transformation agent, protoplast fusion, injection, the electroporation of DNA, the introduction of the DNA by means of the biolistic approach, and other possibilities.

The use of the agrobacteria-mediated transformation of plant cells has been studied intensively and described sufficiently, for example in EP 120 516; Hoekema, in: The Binary Plant Vector System, Offsetdrukkerij Kanters B. V., Alblasserdam (1985), chapter V, 63-71; Fraley et al., Crit. Rev. Plant Sci. 4, 1-46 and in An et al., EMBO J. 4 (2), (1985), 277-284. As regards the transformation of potato, see, for example, Rocha-Sosa et al., EMBO J. 8 (1), (1989), 23-29.

The transformation of monocotyledonous plants by means of vectors based on Agrobacterium has also been described (Chan et al., Plant Mol. Biol. 22 (1993), 491-506; Hiei et al., Plant J. 6 (1994) 271-282; Deng et al., Science in China 33 (1990), 28-34; Wilmink et al., Plant Cell Reports 11 (1992), 76-80; May et al., Bio/Technology 13 (1995), 486-492; Conner and Domisse, Int. J. Plant Sci. 153 (1992), 550-555; Ritchie et al., Transgenic Res. 2 (1993), 252-265). An alternative system for the transformation of monocotyledonous plants is the transformation by means of the biolistic approach (Wan and Lemaux, Plant Physiol. 104 (1994), 37-48; Vasil et al., Bio/Technology 11 (1993), 1553-1558; Ritala et al., Plant Mol. Biol. 24 (1994), 317-325; Spencer et al., Theor. Appl. Genet. 79 (1990), 625-631), protoplast transformation, the electroporation of partially permeabilized cells, or the introduction of DNA by means of glass fibres. In particular the transformation of maize is described repeatedly in the literature (cf., for example, WO 95/06128, EP 0 513 849, EP 0 465 875, EP 0 292 435; Fromm et al., Biotechnology 8 (1990), 833-839; Gordon-Kamm et al., Plant Cell 2 (1990), 603-618; Koziel et al., Biotechnology 11 (1993), 194-200; Mórocz et al., Theor. Appl. Genet. 80 (1990), 721-726).

The successful transformation of other cereal species has also been described, for example for barley (Wan and Lemaux, above; Ritala et al., above; Krens et al., Nature 296 (1982), 72-74) and for wheat (Nehra et al., Plant J. 5 (2), (1994), 285-297; Altpeter et al., Mol. Breeding 6 (2000), 519-528).

The genetically modified cell is regenerated into a plant in accordance with step (b) of the method according to the invention by prior-art methods with which the skilled worker is familiar.

If the expression of the foreign nucleic acid molecule in the plant cells is required for achieving the desired effect, i.e. the reduced activity of the R1 protein, any promoter which is active in plant cells is suitable for this purpose. The promoter can be chosen in such a way that expression in the plants is constitutive or only takes place in a particular tissue, at a particular point in time of plant development or at a point in time determined by external factors. With regard to the plant, the promoter can be homologous or heterologous. Useful promoters are, for example, the cauliflower mosaic virus 35S RNA promoter, the maize ubiquitin promoter and the actin promoter for constitutive expression, or a promoter which ensures expression only in photosynthetically active tissues, for example the ST-LS1 promoter (Stockhaus et al., Proc. Natl. Acad. Sci. USA 84 (1987), 7943-7947; Stockhaus et al., EMBO J. 8 (9), (1989), 2445-2451), the Ca/b promoter (see, for example, U.S. Pat. No. 5,656,496; U.S. Pat. No. 5,639,952; Bansal et al., Proc. Natl. Acad. Sci. USA 89 (1992), 3654-3658) and the Rubisco SSU promoter (see, for example, U.S. Pat. No. 5,034,322 or U.S. Pat. No. 4,962,028), and inducible promoters.

Furthermore, the foreign nucleic acid molecule may contain a termination sequence which serves for the correct termination of transcription and to the addition of a poly-A tail to the transcript, which is thought to have a function in stabilizing the transcripts. Such elements are described in the literature (cf., for example, Gielen et al., EMBO J. 8 (1), (1989), 23-29) and can be exchanged as desired.

The generation of further plants in accordance with step (c) of the method according to the invention can be carried out by any suitable method, for example by vegetative propagation (for example via cuttings, tubers or via callus culture and regeneration of intact plants) or by sexual propagation. Sexual propagation preferably takes place under controlled circumstances, i.e. selected plants which have specific characteristics are hybridized with each other and propagated. The present invention also relates to the plants which can be obtained by this type of propagation

Methods and processes for the generation of transgenic maize plants with a reduced R1 protein activity have already been described in WO 98/27212. In addition to the abovementioned possibilities, the skilled worker can therefore also generate transgenic maize plants with a reduced R1 protein activity as described in WO 98/27212.

While WO 98/27212 describes the generation of transgenic maize plants with a reduced activity of an R1 protein, it does not describe that such transgenic maize plants have an increased leaf starch content. Thus, this publication does not disclose methods for generating maize plants with an increased leaf starch content. Surprisingly, it has now been found that a reduced activity of an R1 protein in maize plants leads to a dramatically increased leaf starch content in the maize plants. Furthermore, such plants surprisingly display the advantage of an improved digestibility of the ensiled material of these plants. This was demonstrated in an artificial system developed for the simulation and study of digestive processes in the rumen of ruminants (Cone et al., Animal Feed Science Technology 61 (1996), 113-128). This system reflects an improved digestibility by an increase in fermentation which, in turn, is reflected in increased gas production. Surprisingly, digestion measurements in such a system which involve the ensiled material of transgenic maize plants with reduced R1 activity reveal a higher total gas production and more rapid gas production rate. This suggests that these plants contain substantially more degradable starch and/or cell-wall material and that, when they are fermented, more of the organic material is broken down. This furthermore allows the conclusion that more starch is broken down in the rumen of ruminants and less starch is transported into the small intestine. Since the total starch intake is limited (approx. 2 kg/day) it is particularly advantageous in feeding if this starch supply is not brought about by increased amounts of feed, but by better conversion of the feed. This constitutes an advantage particularly for example for cattle fattening and for feeding dairy cows late in the lactation period (approx. 10 weeks after calving).

Moreover, the invention encompasses a method for making a maize silage encompassing the step of ensiling transgenic maize plants with a reduced activity of an R1 protein which occurs endogenously in the maize plant.

The present invention thus also relates to a method for making a maize silage in which transgenic maize plants with a reduced activity of an R1 protein are employed. Such plants can be generated for example by the above-described method according to the invention. As regards the definition of the terms and the preferred embodiments, what has already been said in connection with the above-described method according to the invention also applies here.

Silage making is understood as a method for preserving the feed and retaining the feed value characteristics. As a rule, this is done with the exclusion of oxygen.

When making silage starting from maize plants, it is preferred first to comminute the maize plants (chopping to a length of, for example, <10 mm). Moreover, the material to be ensiled may, if appropriate, be bruised or after-treated by means of grating devices in order to ensure high digestibility of the kernels. As a rule, the material to be ensiled is compacted in a clamp without prolonged intermediate storage and the clamp is provided with an essentially air-tight seal. Then, fermentation commences (glucose is broken down into lactic acid and alcohol). The aerobic degradation terminates under anaerobic conditions. The anaerobic degradation is stopped by lowering the pH to underneath the activity limit of the anaerobic microorganisms which are present in the material being ensiled (lactic acid bacteria). As a rule, this is done with the aid of acids which are formed as metabolites of the microorganisms. Silage-making can be done by customary methods with which the skilled worker is familiar, for example as a horizontal arrangement (for example concrete clamps, buried clamps or open clamps), upright, for example in steel or concrete silos, or in the form of big-bale silage (for example film-wrapped bales or silo bags).

An additional possibility is the addition of ensiling agents (for example sugars, lactic acid inoculants), in order to bring about improved stability and/or digestibility of the ensiled material and, if appropriate, prevent mould development.

The present invention furthermore relates to the use of transgenic maize plants with a reduced activity of an R1 protein which occurs endogenously in maize plants for making a maize silage. The important advantage of such a maize silage is the fact that, owing to the increased leaf starch content of the transgenic maize plants, the resulting silage can be utilized considerably better by animals, in particular by ruminants. The efficacy with which such a silage is utilized in the rumen of ruminants is substantially higher. This silage is characterized in particular by the fact that it is utilized better in the rumen of ruminants, preferably of cattle or sheep. The term “utilized better” means that more of the organic material is broken down. By preference, this means that a higher percentage of the starch is broken down in the rumen and less starch is transported into the small intestine.

The better utilization can be determined by first ensiling the plant material in question and subsequently studying the ensiled material in an artificial system which permits the fermentation of feedstuffs in rumen liquid to be studied. In this context, ensiling is preferably carried out as described in the appended Example 5.

The fermentation can preferably be determined as described in Example 6. In such a system, a silage according to the invention displays the characteristic of leading to an increased total gas production in comparison with a silage of corresponding control plants (i.e. corresponding wild-type plants whose R1 protein activity is not reduced). In this context, gas production is preferably increased by at least 10%, preferably by at least 20% and especially preferably by at least 30% in comparison with the gas production of ensiled material of corresponding control plants in the same system. The increase is preferably observed in the second measuring phase (readings A2 to C2).

Moreover, a silage according to the invention has the characteristic of showing a higher gas production rate in a system described in Example 6 in comparison with ensiled material of corresponding control plants, i.e. a greater amount of gas produced per unit time. In preference, the gas production rate is increased by at least 20%, preferably by at least 30% and especially preferably by at least 40%. The increase is preferably observed in the second measurement phase (readings A2 to C2).

Moreover, the present invention relates to a silage which can be obtained by ensiling transgenic maize plants with a reduced activity of an R1 protein which occurs endogenously in the maize plants, or which can be obtained by the method according to the invention for making a maize silage. This maize silage has the above-described characteristics.

The present invention also relates to the use of nucleic acid molecules which encode an R1 protein, or of parts of such nucleic acid molecules, for generating transgenic maize plants with an increased leaf starch content.

The “parts” of such nucleic acid molecules may be, for example, sequence segments which can be employed in connection with the above-described method according to the invention, for example as component of antisense constructs, of ribozymes, of cosuppression constructs, of constructs for in vivo mutagenesis or of constructs for the simultaneous expression of an antisense RNA and a sense RNA which form an RNA duplex, where the constructs in question lead to a reduced activity of an R1 protein in maize plants which have been subjected to relevant genetic modification.

As regards the definitions of the terms and the preferred embodiments, what has been said above in connection with the method according to the invention for generating maize plants with an increased leaf starch content also applies here.

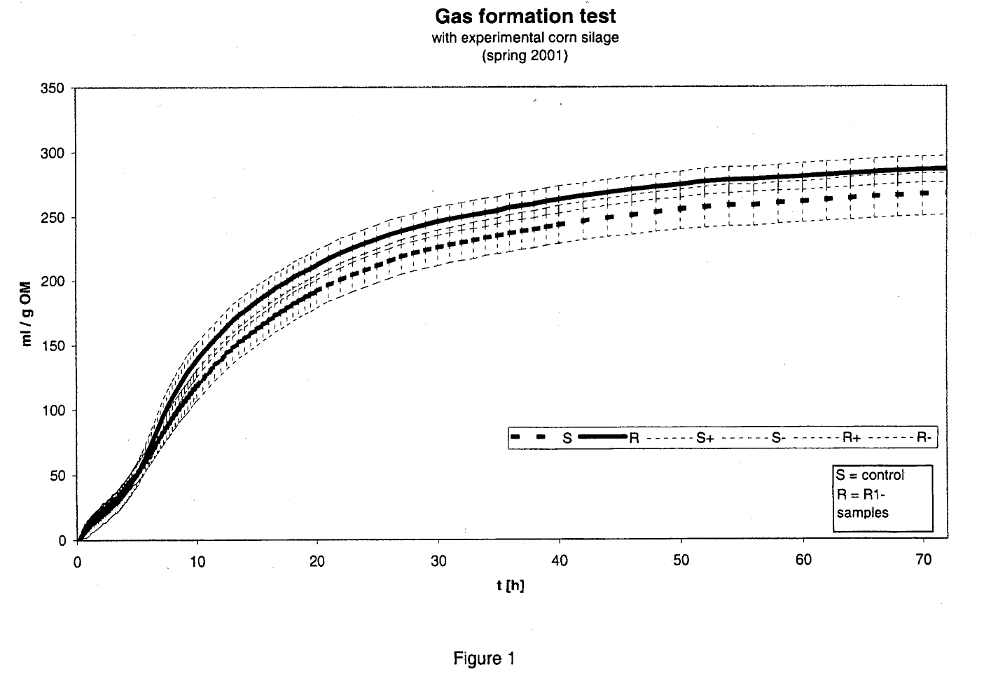

FIG. 1 shows the gas production curve of the breakdown of the organic material (mug OMD) as measured over a period of 72 hours of the controls (S) and of the R1 samples (R) (Example 7).

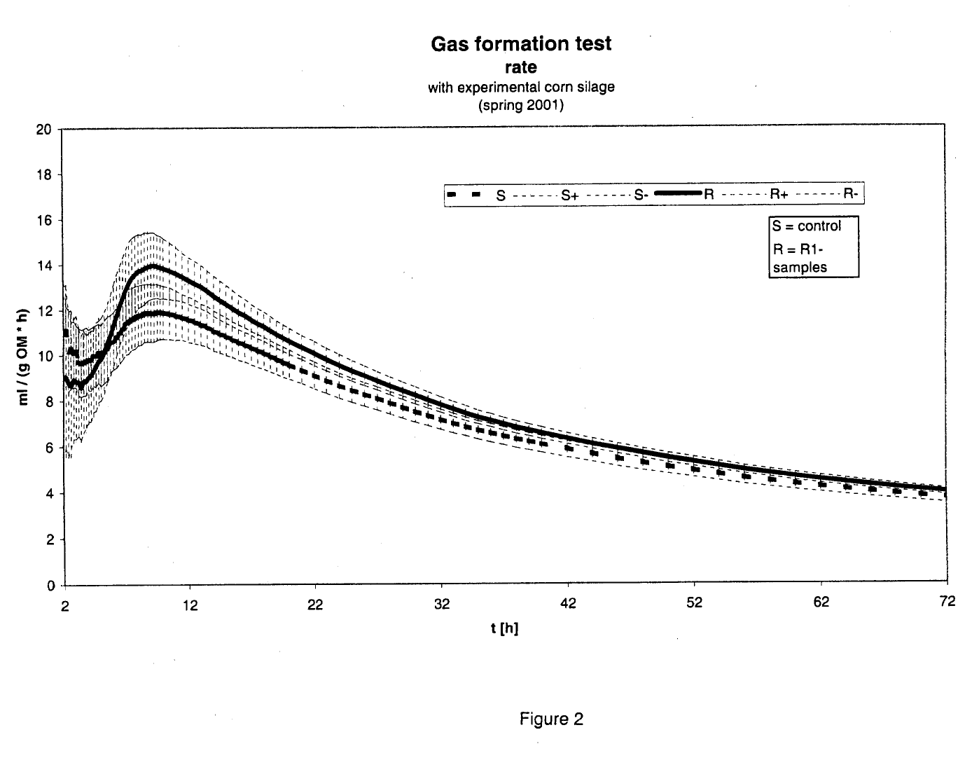

FIG. 2 shows the gas production rate per hour of the curves shown in FIG. 1.

The examples which follow illustrate the invention.

### EXAMPLE 1

Generation of Transgenic Maize Plants

The material used for the transformation was maize protoplasts isolated from highly embryogenic suspension cultures by the method of Mórocz et al. (TAG 80 (1990), 721-726). The transgenic maize plants were generated by destabilizing the membranes by means of polyethylene glycol (PEG) and direct uptake of the DNA fragments into the embryogenic protoplasts via “membrane breakages” (Golovkin et al., Plant Science 90 (1993), 41-52; Omirulleh et al., Plant Mol. Biol. 21 (1993), 415-428). The plasmid employed for the transformation contains the transgene between the T-DNA border sequences. The sequences of the bacterial resistance gene were removed before the transformation by restriction digestion.

The plasmid used for the transformation (IR 23/56) is derived from the cloning vector pUC19 (Yanisch-Peron, Gene 33 (1985), 103-119). To introduce herbicide tolerance to glufosinate ammonium, a pat cassette, which contains the cauliflower mosaic virus CaMV35S promoter sequence for initiating transcription (Franck et al., Cell 21 (1980), 285-294), a synthetic pat gene and the 3′ end signals of the ³⁵S cauliflower mosaic virus (Franck et al., loc. cit.) was cloned. The pat gene encodes a synthetic gene version of the phosphinothricin acetyltransferase enzyme (PAT), which was originally isolated from Streptomyces viridochromogenes.

Thereafter, the 2.2 kb XhoI fragment of the maize R1 cDNA (corresponds to position 2125-4324 of the sequence shown in SEQ ID NO: 1) was inserted in sense orientation into the XhoI restriction site of the vector. The maize ubiquitin promoter (Christensen et al., Plant Mol. Biol. 18 (1992), 675-689) was employed for constitutive expression; to increase gene expression, it was cloned upstream. The signals for transcriptional termination and for polyadenylation were amplified from the pTiT37 T-DNA (Depicker et al., Journal of Molecular and Applied Genetics 1 (1982), 561-573) and cloned downstream of the partial R1 cDNA.

To transform maize, the plasmid IR23/56 was digested with the restriction enzyme ApaLI in combination with FseI, SwaI or AscI. The fragment with a length of over 5700 bp, which contains the relevant segments, was used for the transformation. Intact plants were regenerated from transformed cells.

### EXAMPLE 2

Determination of the Starch Content of Leaf Material

(a) Sample Preparation:

The material used for determining the starch content was fully-grown leaves of 8-10-week-old plants which had been grown under constant light conditions (400 μE).

The leaves were harvested at midday.

Removal of the Soluble Sugars by Extraction with Ethanol:

Approx. 1 g of fresh leaf material from the transgenic plants generated as described in Example 1 was freeze-dried, weighed and subsequently homogenized to a fine powder using a Retsch ball mill. Approx. 50 mg of powdered leaf material (determination in duplicate) were weighed, 1 ml of 80% strength ethanol was added, the mixture was shaken vigorously, and the homogeneous dispersion was incubated for 1 h in a water bath at 80° C. After the dispersion had cooled to approx. 40° C., it was centrifuged for 5 min at 3 000 rpm (Minifuge RF, Heraeus). The supernatant was discarded. The leaf material was treated twice more with in each case 1 ml of 80% strength ethanol and incubated for in each case 20 min in a water bath at 80° C. After cooling and centrifuging (see above), all the supernatants were discarded.

(b) Starch Determination in a Microtitre Plate/Spectramax at 340 nm:

Stärke Lebensmiffelanalytik UV-Test, Boehringer Mannheim, Catalogue No.: 207748 (amyloglucosidase, starch determination buffer, glucose-6-phosphate dehydrogenase)

The sugar-free leaf material is treated with 400 microlitres 0.2 N KOH and homogenized by shaking vigorously. The homogenate is incubated for 1 h at 95° C. in a water bath. After cooling, 75 μl 1M acetic acid are added and the reaction mixture is mixed thoroughly. The mixture is centrifuged for 10 min at 4 000 rpm. 25 and 50 μl supernatant are introduced into a microtitre plate containing 50 μl amyloglucosidase (Boehringer Mannheim) and 25 or 50 μl, respectively, of Millipore water and digested for 1 h at 56° C. 196 μl starch determination buffer (Boehringer Mannheim) are introduced into another microtitre plate. To this there are added 4 (to 20) μl of the cooled starch digest. The ratio can be raised to up to 40 μl digest+160 μl starch determination buffer, depending on the glucose concentration.

| | |
|---|---|
| Measurement: | shake, pre-read |
| | +2 μl glucose-6-phosphate dehydrogenase |
| | (Boehringer Mannheim) |
| | incubation: 30 min at 37° C., measure |

(c) The Starch Content was Calculated as Follows:
Measuring volume(200 μl)×extraction volume(4 750 μl)×amyloglucosidase digest volume(200 μl)×ΔOD/ε×1 000× sample measurement volume(4 μl . . . 1.40 μl)×sample digest volume(50 μl)×weight (g)×d(1)=concentration(μmol/g DW)

ε=6.3 l×mmol⁻¹×cm⁻¹ (molar extinction coefficient of NADH at 340 nm)

DW=dry weight

The concentration in mg glucose/g fresh weight was calculated from the determined weights before and after freeze-drying and the molecular weight of glucose (162.1 g/mol—anhydride).

### EXAMPLE 3

Leaf Starch Extraction

Reagents

| | |
|---|---|
| Extraction buffer | 50 mM Na-MOPS |
| pH 7.3: | MOPS = (3-[N-morpholino]propanesulphonic acid) |
| | 2 mM EDTA |
| | 0.5 mM beta-mercaptoethanol |
| SDS 2% (Serva) | |
| ethanol 80% | |
| acetone | |

Starch Extraction

Using a Waring blender, the leaves of the plants were comminuted for approx. three minutes at the highest speed, using extraction buffer (8 ml per gram fresh weight). The mixture is subsequently filtered first through a kitchen strainer and then through 125μ filter. The solids are again homogenized in the Waring blender using extraction buffer (2 ml per gram fresh weight) and again filtered. After the second extraction, the solids are discarded. The filtrates are combined in a centrifuge bottle and centrifuged for 15 min at 5 500×g. The supernatant is discarded and the pellet is taken up in 2% SDS (8 ml per gram fresh weight). The suspension is filtered through a 30μ filter with gentle stirring (starch passes through the filter) and then centrifuged for 15 min at 5 500×g. The supernatant is discarded and the pellet is washed three times with water (8 ml per gram fresh weight; resuspended and centrifuged as described above), and all the supernatants are discarded. Again, the pellet is taken up in water (8 ml per gram fresh weight) and the mixture is again filtered through a 30μ filter, if possible without stirring.

The filtrate is subsequently centrifuged for 15 min at 5 500×g and the pellet is washed twice with 80% ethanol and once with acetone (in each case 0.5 ml per gram fresh weight; resuspended and centrifuged as described above).

After a final wash with water (0.5 ml per gram fresh weight), the pellet (leaf starch) is dried for at least 24 h in a lyophylizer.

Phosphate Determination

Reagents:

| 0.7 N HCl: | 2.9 ml 37% strength HCl/50 ml | | |
|---|---|---|---|
| Buffer: | 9 | ml | 1 M imidazole solution pH 7.2 |
| | 225 | μl | 1 M MgCl₂ |
| | 60 | μl | 0.5 M EDTA |
| | 150 | μl | 80 mM NADP |
| | 20.565 | ml | Millipore water |
| | 30.0 | ml | |

| | |
|---|---|
| Enzyme: | Glucose-6-phosphate dehydrogenase (from Leuconostoc |
| | mesenteroides) Roche, Catalogue No.: 165875, |
| | 1 000 U/1 ml, dilute 1:4 with buffer |

Sample Preparation:

100 mg leaf starch (weighed accurately) are weighed into a 2 ml Safe-Look Eppendorf tube and treated with 500 μl of 0.7 N HCl. During weighing, the water content of a further 100 mg of leaf starch is determined by means of a temperature-controlled balance.

The mixture is vortexed vigorously; this is followed by acid hydrolysis for 4 hours at 95° C. with shaking. After cooling, the mixture is centrifuged for 20 min at 13 000 rpm. All of the supernatant is transferred into a Spin Module Size 100 (Q.BIOgene) and filtered by briefly centrifuging.

140 μl of hydrolysate are mixed with 1 260 μl of buffer in the Eppendorf tube, and two 700 μl aliquots are transferred into two quartz cuvettes (reference cuvette and sample cuvette). Enzymatic determination of glucose-6-phosphate is started by addition of 6 μl of enzyme to the sample cuvette. The measurement (increase in NADPH) is carried out at 340 nm using a UVIKON apparatus (Kontron).
Calculation:

$$\frac{\begin{matrix}Measuring volume ( 700 μ l ) \times \\ extraction volume ( 500 μ l ) \times deltaOD\end{matrix}}{ɛ \times sample volume ( 70 μ l ) \times {weight}^{*} ( mg ) \times d ( = 1 )} = C 6 P concentration per dry leaf starch in nmol \text{/} mg$$

ε=6.3 l×mmol⁻¹×cm⁻¹ (molar extinction coefficient of NADPH at 340 nm)
d=path length of the cuvette

### EXAMPLE 4

Result of the Leaf Starch Content Determination

The leaf starch content determination of the plants generated as described in Example 1 gave the following results.

TABLE 1
Leaf starch contents of the plants employed
(R1 plants or controls, averaged)

| | | Water | | mg | | mg starch |
|---|---|---|---|---|---|---|
| | Leaf | content | | starch/g | Starch | (DW)/g |
| Material | FW/g | of starch | Starch/mg | leaf FW | (DW)/g | Leaf FW |
| R1 plants | 182 | 8.81% | 1189.5 | 6.54 | 1084.71 | 5.96 |
| Controls | 154 | 19.70% | 49.00 | 0.32 | 39.35 | 0.26 |

FW = Fresh weight
DW = Dry weight

### EXAMPLE 5

Work-Up of the Plant Material and Silage Making

The maize plants used were isogenic material originating from the same cross. Crosses were performed between Event, which is transgenic, and an old land race (=wild type). The Basta-resistant (=R1) plants and the Basta-sensitive plants (=controls, referred to as “SiC”) were selected. The genetic background was thus identical, with the exception of the transgene.

In one set-up, two isogenic comparable sets of Basta-resistant maize lines (increased leaf starch content) and sensitive maize lines (F74T46) with adjusted ratios between cob biomass (see tables—FW=fresh weight/cob FW=cob fresh weight) to total plant biomass were shredded with the aid of an all-purpose garden shredder Natura 2800 L (Gloria).

For setting up the experimental silage, tubular, hermetically sealable minisilos were used. The material used was grey HD plastic tubes with a length of 45 cm and a diameter of 15 cm. They were sealed with plastic lids on either end and, after filling, stored horizontally. Each silo was filled stepwise in portions of approximately ¼ of the filling volume and the filling material was compressed using a weight. After filling of the silos had ended, they were sealed hermetically and stored in the sealed state for 15 weeks at room temperature (T=15°-20° C.).

In total, 6 minisilos were studied, viz. three controls (Basta-sensitive maize) and three R1 samples (Basta-resistant maize with increased leaf starch content).

| | | | of which cob |
|---|---|---|---|
| | | | material in kg |
| Material | Sample name | Weight (FW) in kg | (= %) |
| Control | 130 S1C | 3.79 | 1.23 (30%) |
| | 121 S2C | 4.11 | 1.28 (32%) |
| | 101 S3C | 1.8 | 0.56 (32%) |
| R1 plants | 120 R1 | 3.79 | 1.19 (29%) |
| | 110 R2 | 4.10 | 1.28 (32%) |
| | 98 R3 | 1.8 | 0.56 (32%) |

### EXAMPLE 6

Silage Gas Measurements

A representative sample was taken from each of the six minisilos, dried at 70° C. and screened through a 1 mm screen. The dried samples were analysed for dry matter and ash content. 0.5 gram of the air-dried material was incubated in the gas production analyser. The gas production was measured over 72 h in a buffer containing 32% rumen fluid from 2 cows (Cone et al., Animal Feed Science Technology 61 (1996), 113-128). This techniques enables the fully automated measurement of the gas production; it involves the use of a transducer and electric micro-valves.

This system makes it possible to measure the fermentation kinetics of animal food in an in vitro system using rumen microorganisms by incubating the feedstuffs in a buffered rumen liquid. The feedstuff is incubated in sealed vessels which are connected to the gas production plant. During the incubation, the pressure in the vessel rises as a function of gas formation. The gas production system records any change in pressure, and a certain degree of superatmospheric pressure triggers a signal which allows one of the valves to open. When a defined amount of gas has escaped, the valves shut again. The duration during which the valves are open is recorded by the analytical unit. The stored data can be fed into a PC, with which further subsequent calculations can be carried out.

Each unit is capable of measuring 12 incubations simultaneously; moreover, 10 units can be connected to one PC.

This gas production technique makes possible the combination of a plurality of measurement opportunities during incubation and thus the recording of minute differences in the fermentation characteristic.

The gas production curves were represented as described by Cone et al. (1996, loc. cit.), by Cone et al. (Anim. Sci. Feed Technol. 66 (1997), 31-45) and Groot et al. (Anim. Feed Sci. Technol. 64 (1996), 77-89). In this model, several phases can be distinguished in a gas production curve. Each phase in turn can be described by three parameters. Frequently, gas production is divided into three phases, so that an overall curve can be described by nine parameters.
GP(t)=a₁/(1+(b₁/t)^{c1})+a₂/(1+(b₂/t)^{c2})+a₃/(1+(b₃/t)^{c3})

In this formula,

GP(t)=gas production at time t (ml/g biomass),

aᵢ=maximum gas production of phase i (ml),

bᵢ=time in which half of the gas production aᵢ is reached (in hours), and

cᵢ=shape parameter (a higher value indicates a steeper slope)

Phase 1 describes the breakdown of the rapidly fermentable, soluble material during the first hours of incubation. Phase 2 describes the breakdown of NDF and starch, if present, over a period of up to 20 hours. Gas production after over 20 hours is referred to as Phase 3. This phase is brought about by the fermentation of material which is digested very slowly and by microbial decomposition in the in vitro system (Cone et al., (1997), loc. cit.). The third phase is of less importance for evaluating feedstuffs. A fractional substrate digestion can be calculated in each phase using the above-described model. This value (R) climbs to a maximum after a specific time tRmax (hours), as described by Groot et al. (loc. cit.).

In silage samples, most of the gas production is coupled with the breakdown of cell wall material and of starch, which makes the second phase the most important phase for evaluating feedstuffs. A change in the gas production profile can be seen most clearly in the amount of ml (a2) and the half life (b2), but also in the shape (c2). Both the value tRmax of the second phase, calculated from b2 and c2, and the value R, likewise calculated from b2 and c2 and tRmax of the same phase, are readily distinguishable characteristics for the breakdown of a sample. A higher value R, frequently in combination with an earlier tRmax value, represents a sample which is broken down more readily.

In addition to the gas production profile, the breakdown rate of organic material (OMD=organic matter disappearance, g/kg OM) after 72 hours' incubation was measured. This value is frequently closely related to the total gas production after 72 hours (GP72, ml/g organic material).

### EXAMPLE 7

Results Obtained with the Silage Samples

Table 2 shows the dry matter (g/kg fresh weight) and the ash content (g/kg dry matter) of the test samples.

TABLE 2
Dry matter (DM; g/kg fresh weight)
and ash content (g/kg dry matter)

| Sample | DM | Ash |
|---|---|---|
| 130 S1C | 199 | 65 |
| 121 S2C | 196 | 59 |
| 101 S3C | 152 | 74 |
| 120 R1 | 189 | 66 |
| 110 R2 | 201 | 63 |
| 98 R3 | 180 | 69 |

It can be seen from the table that the dry matter and the ash content differ somewhat between different silage samples (even though the samples are two triplicates).

Table 3 shows the results of the gas production parameter measurements. Table 3 encompasses the total gas production after 72 hours' incubation and the OMD value after 72 hours' incubation. Likewise, the calculated maximum relative breakdown rate of the second phase (Rmax2) and the point in time at which it took place (tRmax2) were calculated.

TABLE 3
Gas production parameter measurements, means of the S and R samples, and
P values for the differences between the means of the S and R samples.

| Sample | A1 | B1 | C1 | A2 | B2 | C2 | A3 | B3 | C3 |
|---|---|---|---|---|---|---|---|---|---|
| 130 S1C | 29 | 1.6 | 0.94 | 174 | 8.9 | 2.69 | 87 | 30.4 | 2.9 |
| 121 S2C | 36 | 1.5 | 1.25 | 160 | 9.6 | 2.46 | 83 | 29.11 | 2.5 |
| 101 S3C | 26 | 1.7 | 1.33 | 150 | 10.1 | 2.63 | 81 | 29.9 | 2.5 |
| 120 R1 | 15 | 2.0 | 1.61 | 181 | 9.2 | 3.06 | 85 | 31.5 | 3.3 |
| 110 R2 | 29 | 1.5 | 0.97 | 187 | 8.6 | 2.85 | 77 | 30.0 | 3.1 |
| 98 R3 | 31 | 1.7 | 0.82 | 188 | 8.3 | 2.85 | 79 | 29.5 | 3.1 |
| S | 30 | 1.6 | 1.17 | 161 | 9.5 | 2.59 | 84 | 29.8 | 2.6 |
| R | 25 | 1.7 | 1.13 | 185 | 8.7 | 2.92 | 80 | 30.3 | 3.2 |
| P value | 0.41 | 0.44 | 0.89 | 0.03 | 0.13 | 0.03 | 0.33 | 0.50 | 0.02 |

The data shown here show the three breakdown phases 1-3. The gas production of the R1 silage samples is 13% higher in only 97% of the time, in comparison with the controls, in particular during the second phase (values A2, B2 and C2), during which the starch and the NDF (neutral detergent fibres) are broken down (up to approx. 20 h post-incubation).

TABLE 4
Measured breakdown of the organic material (OMD, g/kg), total
gas production after 72 hours' incubation (GP72, ml/g organic
material), maximum relative breakdown rate of the second phase
(Rmax2), point in time at which it took place (tRmax2, hours),
means of the S and R samples, and P values for the differences
between the means of the S and R samples.

| Sample | OMD | GP72 | Rmax2 | TRmax2 |
|---|---|---|---|---|
| 130 S1C | 789 | 282 | 0.156 | 10.8 |
| 121 S2C | 784 | 271 | 0.130 | 11.2 |
| 101 S3C | 732 | 249 | 0.135 | 12.1 |
| 120 R1 | 772 | 271 | 0.178 | 11.6 |
| 110 R2 | 802 | 289 | 0.173 | 10.7 |
| 98 R3 | 813 | 292 | 0.179 | 10.3 |
| S | 768 | 267 | 0.140 | 11.4 |
| R | 796 | 284 | 0.177 | 10.9 |
| P value | 0.28 | 0.23 | 0.01 | 0.41 |

Over the entire 72 hour period, the R1 silage samples revealed not only a 3.6% higher breakdown rate of the organic material (OMD=796 g/kg in comparison with 768 g/kg of the control), but also a 6% higher gas production (GP72=284 ml/g in comparison with 267 ml/g organic material in the controls). In the second phase, in particular, the maximum relative breakdown rate (Rmax2) of the R1 silage markedly (=26%) outperforms that of the control and took place within less time, i.e. 96% of the time.

Based on a higher gas production after 72 hours' incubation and a higher breakdown rate of organic material, the digestibility of the R1 silage can be considered to be better. The transgenic R1 silage shows a significantly higher amount of broken down starch and cell wall material.

The curves of the means are shown in FIG. 1 (=gas formation test 1). FIG. 2 shows the gas production rate per hour of the same curves (=gas formation test 2).

1. A method for generating a transgenic maize plant with an increased starch content in comparison to corresponding wild-type plants, where
(a) a cell of a maize plant is genetically modified by introducing a foreign nucleic acid molecule whose presence or expression leads to a reduced activity of an R1 protein which occurs endogenously in the plant cell;
(b) a plant is regenerated from the cell generated in step (a);
(c) if appropriate, further plants are generated starting from the plant generated in step (b); and
(d) the foreign nucleic acid molecule according to step (a) is a DNA molecule which encodes at least one antisense RNA which brings about reduced expression of endogenous genes which encode R1 proteins.

2. The method according to claim 1, wherein the foreign nucleic acid molecule according to step (a) is a DNA molecule which, via a cosuppression effect, leads to reduced expression of endogenous genes which encode R1 proteins.

3. The method according to claim 1, wherein the foreign nucleic acid molecule according to step (a) is a DNA molecule which encode at least one ribozyme which specifically cleaves transcripts of endogenous genes which encode R1 proteins.

4. The method according to claim 1, wherein the foreign nucleic acid molecule according to step (a) is selected from the group consisting of nucleic acid molecules which, in the event of in vivo mutagenesis, lead to a mutation or an insertion of a heterologous sequence in endogenous genes which encode R1 proteins, the mutation or insertion leading to reduced expression of genes encoding R1 proteins, or to a reduced synthesis of active R1 proteins.

5. The method according to claim 1, wherein the foreign nucleic acid molecule according to step (a) is a DNA molecule which simultaneously encodes at least one antisense RNA and at least one sense RNA, where the said antisense RNA and the said sense RNA form an RNA duplex which brings about a reduced expression of endogenous genes which encode R1 proteins.

6. Method according to claim 1, where the reduced activity of the R1 protein means an amount of R1 protein which is reduced by at least 50% in comparison with corresponding cells which have not been genetically modified.

7. A method for making a silage, comprising the step of ensiling transgenic maize plants with a reduced activity of an endogenous R1 protein where the transgenic maize plants were generated by the following steps:
(a) a cell of a maize plant is genetically modified by introducing a foreign nucleic acid molecule whose presence or expression leads to a reduced activity of an R1 protein which occurs endogenously in the plant cell;
(b) a plant is regenerated from the cell generated in step (a);
(c) if appropriate, further plants are generated starting from the plant generated in step (b); and
(d) the foreign nucleic acid molecule according to step (a) is a DNA molecule which encodes at least one antisense RNA which brings about reduced expression of endogenous genes which encode R1 proteins.

8. The method according to claim 7, wherein the foreign nucleic acid molecule according to step (a) is a DNA molecule which, via a cosuppression effect, leads to reduced expression of endogenous genes which encode R1 proteins.

9. The method according to claim 7, wherein the foreign nucleic acid molecule according to step (a) is a DNA molecule which encode at least one ribozyme which specifically cleaves transcripts of endogenous genes which encode R1 proteins.

10. The method according to claim 7, wherein the foreign nucleic acid molecule according to step (a) is selected from the group consisting of nucleic acid molecules which, in the event of in vivo mutagenesis, lead to a mutation or an insertion of a heterologous sequence in endogenous genes which encode R1 proteins, the mutation or insertion leading to reduced expression of genes encoding R1 proteins, or to a reduced synthesis of active R1 proteins.

11. The method according to claim 7, wherein the foreign nucleic acid molecule according to step (a) is a DNA molecule which simultaneously encodes at least one antisense RNA and at least one sense RNA, where the said antisense RNA and the said sense RNA form an RNA duplex which brings about a reduced expression of endogenous genes which encode R1 proteins.

12. A method for making silage which comprises ensuing transgenic maize plants with a reduced activity of an endogenous R1 protein where the reduced activity is caused by a DNA molecule which encodes at least one antisense RNA.

13. The method according to claim 12, wherein the reduced activity is caused by a DNA molecule which, via a cosuppression effect, leads to reduced expression of endogenous genes which encode R1 proteins.

14. The method according to claim 12, wherein the reduced activity is caused by a DNA molecule which encodes at least one ribozyme which specifically cleaves transcripts of endogenous genes which encode R1 proteins.

15. The method according to claim 12, wherein the reduced activity is caused by nucleic acid molecules which, in the event of in vivo mutagenesis, lead to a mutation or an insertion of a heterologous sequence in endogenous genes which encode R1 proteins, the mutation or insertion leading to reduced expression of genes encoding R1 proteins, or to a reduced synthesis of active R1 proteins.

16. The method according to claim 12, wherein the reduced activity is caused a DNA molecule which simultaneously encodes at least one antisense RNA and at least one sense RNA, where the said antisense RNA and the said sense RNA form an RNA duplex which brings about a reduced expression of endogenous genes which encode R1 proteins.

17. Silage which is produced by the process which comprises silage ensiling transgenic maize plants with a reduced activity of an endogenous R1 protein where the reduced activity is caused by a DNA molecule which encodes at least one antisense RNA.

18. The silage according to claim 17, wherein the reduced activity is caused by a DNA molecule which, via a cosuppression effect, leads to reduced expression of endogenous genes which encode R1 proteins.

19. The silage according to claim 17, wherein the reduced activity is caused by a DNA molecule which encodes at least one ribozyme which specifically cleaves transcripts of endogenous genes which encode R1 proteins.

20. The silage according to claim 17, wherein the reduced activity is caused by nucleic acid molecules which, in the event of in vivo mutagenesis, lead to a mutation or an insertion of a heterologous sequence in endogenous genes which encode R1 proteins, the mutation or insertion leading to reduced expression of genes encoding R1 proteins, or to a reduced synthesis of active R1 proteins.

21. The silage according to claim 17, wherein the reduced activity is caused a DNA molecule which simultaneously encodes at least one antisense RNA and at least one sense RNA, where the said antisense RNA and the said sense RNA form an RNA duplex which brings about a reduced expression of endogenous genes which encode R1 proteins.

22. The method according to claim 1, wherein the foreign nucleic acid molecule comprises the coding region of the nucleotide sequence shown in SEQ ID NO: 1.

23. The method according to claim 1, wherein the foreign nucleic acid molecule encodes a protein comprising the amino acid sequence shown in SEQ ID NO: 2.

24. The method according to claim 1, wherein the foreign nucleic acid molecule has at least 80% homology with the nucleic acid molecules shown in SEQ. ID NO: 1 or SEQ ID NO:2.

25. The method according to claim 1, wherein the foreign nucleic acid molecule is degenerated in comparison with the sequences of the nucleic acid molecules shown in SEQ. ID NO: 1 or SEQ ID NO:2 or the sequence of the nucleic acid molecule having at least 80% homology with the nucleic acid molecules shown in SEQ. ID NO: 1 or SEQ ID NO:2.
